(19) United States
(12) Patent Application Publication
(10) Pub. No.: US 2025/0049590 A1
(43) Pub. Date: Feb. 13, 2025

(54) ARTIFICIAL KNEE JOINT, AND METHOD FOR CONTROLLING SAME

(71) Applicant: OTTOBOCK SE & CO. KGAA, Duderstadt (DE)

(72) Inventors: Martin WEBER, Duderstadt (DE); Roland AUBERGER, Duderstadt (DE); Robert HOFFMANN, Duderstadt (DE); Viktor Gerhard HÖRIG, Duderstadt (DE); Jens NÖRTHEMANN, Duderstadt (DE); Andreas SCHUH, Duderstadt (DE); Olaf KROLL-ORY-WAHL, Duderstadt (DE)

(21) Appl. No.: 18/719,019

(22) PCT Filed: Dec. 12, 2022
(86) PCT No.: PCT/EP2022/085367

§ 371 Date: Jun. 12, 2024

(30) Foreign Application Priority Data
Dec. 13, 2021 (DE) 10 2021 006 128.4

(51) Int. Cl. A61F 2/74; A61F 2/50; A61F 2/64; A61F 2/68; A61F 2/70; A61H 1/02; A61H 3/00

(52) U.S. Cl. CPC A61F 2/748; A61F 2/64; A61F 2/741; A61H 1/024; A61H 3/00; A61F 2002/5006; A61F 2002/6863; A61F 2002/701; A61H 2201/1246; A61H 2201/5051

(57) ABSTRACT

The invention relates to an artificial knee joint comprising; an upper part (10) and a lower part (20) which are mounted on one another such that they can pivot about a pivot axis (12); a hydraulic resistance device (30) between the upper part (10) and the lower part (20), which resistance device provides resistance to a pivoting movement, the resistance device (30) having a switching valve (50) in a hydraulic line (37), the switching valve (50) having a valve body which can be displaced in a displacement direction and, in a first position, blocks or partially closes the hydraulic line (37) and, in a second position, releases the hydraulic line (37) and is designed or positioned in such a manner that a pressure force component acting on the valve body (55) perpendicularly to the displacement direction through the hydraulic fluid generates a holding force that counteracts a displacement of the valve body (55), wherein an actuator (60) for exerting a release force is associated with the valve body (55) and moves the valve body (55) from the first to the second position, characterized in that the actuator (60) is coupled to a control device (70) which is connected to a sensor for detecting state data and activates the actuator (60) on the basis of the state data, and the release force is set to be less than the holding force at a predefined pressure force.

The invention relates to an artificial knee joint having an upper part and a lower part, which are mounted on one another so that they can pivot about a pivot axis, having a hydraulic resistance device between the upper part and the lower part, by means of which a resistance to a pivoting movement is provided, the resistance device having a switching valve in a hydraulic line, the switching device having a valve body which can be displaced in a displacement direction, blocks or partially closes the hydraulic line in a first setting, particularly in order to close the hydraulic line substantially or fully so as to achieve a throttle effect or blockage, and releases the hydraulic line in a second setting, and is configured or arranged in such a way that a pressure force component acting on the valve body perpendicularly to the displacement direction generates a holding force which opposes a displacement of the valve body. The invention likewise relates to a method for controlling such an artificial knee joint. Artificial knee joints are used in orthoses or prostheses. The hydraulic resistance device may be configured both as linear hydraulics and as rotational hydraulics. The linear hydraulics or rotational hydraulics may also use a magnetorheological liquid as the hydraulic fluid, in order to provide an additional possibility for modulating the properties of the resistance device.

In order to provide adaptation of a resistance against an extension movement and/or flexion movement while walking and standing, or during other activities and states, the artificial knee joint is assigned resistance devices, or at least one resistance device. This resistance device can vary the respective resistance. Various resistance devices are used for this purpose: besides purely mechanical braking devices or locking mechanisms, it is also possible to use magnetorheological resistance devices, electric motors in generator operation, pneumatic dampers or hydraulic resistance devices. The variation of the resistance may take place on the basis of sensor data, which are recorded by means of sensors and are evaluated in a control device. The resistance is then increased or reduced on the basis of the sensor data. This may lead to a turning movement being braked or blocked, or the resistance being varied. Sensor data are in particular the spatial orientation of components, active forces, moments, temperatures, speeds and/or accelerations. A further possibility for varying the resistance consists in using mechanical devices which cause an increase or reduction of the respective resistance as a function of forces, moments or positions, in particular angular settings. For this purpose, for example, throttles in hydraulic devices are adapted and/or flow channels are enabled or closed.

Purely electromechanical control is comparatively expensive and requires sensor values that are as unambiguous as possible in order to ascertain the respective state of the prosthesis or orthosis and the current gait and the walking situation. Particularly for knee joints, it is necessary to prevent an incorrect reduction of flexion resistances in order to prevent unintended bending and collapse of the joint.

It is therefore an object of the present invention to provide an economical artificial knee joint having an adaptable resistance device for safeguarding the stance phase, which reliably prevents erroneous initiation of the swing phase with the associated resistance reduction in dangerous situations.

This object is achieved by an artificial knee joint having the features of the main claim and by a method having the features of the alternative independent claim. Advantageous embodiments and developments of the invention are explained in the dependent claims, the description and the figures.

The artificial knee joint having an upper part and a lower part, which are mounted on one another so that they can pivot about a pivot axis, having a hydraulic resistance device between the upper part and the lower part, by means of which a resistance to a pivoting movement is provided, the resistance device having a switching valve in a hydraulic line, the switching device having a valve body which can be displaced in a displacement direction, blocks the hydraulic line in a first setting and releases the hydraulic line in a second setting, and is configured or arranged in such a way that a pressure force component acting on the valve body perpendicularly to the displacement direction as a result of the hydraulic fluid generates a holding force which opposes a displacement of the valve body, wherein an actuator for exerting a release force which moves the valve body out of the first setting into the second setting is assigned to the valve body. The actuator is coupled to a control device which is connected to a sensor for recording status data and activates the actuator on the basis of the status data, the release force being adjusted to be less than the holding force for a predefined pressure force. The valve body arranged inside the hydraulic line can at least partially block, in particular fully block, the hydraulic line inside the hydraulic system of the hydraulic resistance device so that the hydraulic fluid does not flow in the hydraulic line, or can only flow along with a high resistance. The hydraulic line is in particular a connecting line between two hydraulic chambers, which are separated from one another by a piston in a hydraulic damper. The valve body is mounted inside the switching valve, the mounting and/or the valve body being configured in such a way that a pressure force component acting perpendicularly to the displacement direction is generated when a pressure prevails in the hydraulic line. This pressure force component presses the valve body into its valve seat, particularly in the closed setting. The configuration of the valve body may also be such that a force component directed into the first setting, or closed setting, is exerted by the hydraulic fluid, for example through an obliquely oriented incident flow surface of the valve body. The holding force is applied, for example, by a movement performed transversely with respect to the displacement direction and pressing against the valve seat. Friction forces that occur, or correspondingly shaped form-fit elements, prevent the valve body being displaced from the first setting into the second setting or release setting when a pressure is applied by the hydraulic fluid. In addition to this configuration, the valve body is assigned an actuator which exerts a release force on the valve body and moves the latter from the first setting into the second setting. This actuator is coupled to a control device which activates the actuator, in order to move the switching valve with the valve body from the first setting into the second setting, on the basis of sensor data, for example in respect of the spatial orientation, acceleration, angular velocities or forces, in particular of the lower part or of the upper part or of orthotic or prosthetic components arranged thereon. So long as no hydraulic pressure, or only a small hydraulic pressure, acts on the valve body, adaptation is possible by means of the actuator with corresponding sensor data. The release force, however, is adjusted to be less than the holding force for a predefined pressure force. If the pressure level inside the hydraulic line exceeds a particular limit value, the actuator is no longer capable of moving the switching valve from the locked setting into the release setting. This prevents unintended release of the switching valve taking place and turning of the knee joint thereby being enabled beyond a defined loading of the knee joint, for example in the event of bending, in the locked state due to erroneous or inaccurate sensor values.

In a further embodiment, the valve may be configured in such a way that, after it has been switched from the first setting into the second setting, the valve remains in the second setting so long as the flow rate in the valve does not fall below a defined value. If the flow ceases or changes its direction, the valve automatically returns into the first setting and closes the channel. This may, for example, be achieved with a valve design in which an incident flow surface of the valve body is aligned so that a force component acting in the direction of the second setting in the displacement direction is generated. The incident flow surface may, for example, be set obliquely or the incident flow may be guided in a corresponding direction or at a corresponding angle onto the valve body in order to maintain an opened or released setting. By virtue of these special valve properties, after the valve has been switched into the second setting in order to initiate the swing phase, a knee flexion is possible with low resistance. As soon as the movement is reversed and the knee joint carries out an extension movement, the valve returns into the first setting so that a high resistance against knee flexion is provided. The extension movement is still possible because of a nonreturn valve connected in parallel, since the fluid can flow substantially unimpeded through the nonreturn valve during movement in the extension direction. By this special valve configuration, protection against stumbling may be provided without a sensor system for recording the knee movement being necessary.

As an alternative to this special valve embodiment, protection against stumbling may also be provided by taking into account the knee angle and/or knee angular velocity measured by corresponding sensors. In this case, the valve is kept open by the control unit until the aforementioned sensors detect a movement reversal or an end of the swing phase.

The actuator is configured in particular as a motor or as an electromagnet, which is activated by the control device on the basis of a corresponding control signal. The electric motor may be coupled to the valve body by means of a gearing, in particular an adjustable gearing, a lever mechanism or a link rod combination. The adaptable electromagnet or the solenoid may likewise act directly on the valve body or on the valve body via a force transmission element, a lever gearing or a connecting rod mechanism.

Linear or rotational actuation may be provided both for the valve design and for the actuator system. A linear movement may be converted into a rotational movement, and vice versa, by corresponding lever mechanisms.

In one embodiment, the valve body is assigned a spring element, or energy storage mechanism, which opposes the release force. Alternatively or in addition, a magnet which opposes the release force or a correspondingly engaged magnet may be assigned to the valve body in order to adjust a force threshold beyond which the actuator can move the valve body into the release setting. This is advantageous when the pressure force applied by the hydraulic fluid is not sufficient to generate a sufficient holding force or a desired holding force.

In one embodiment, the release force that the actuator can apply is adjustable, for example by corresponding activation of a coil, by adaptation of a gearing, a drive power of a motor, the prestress of a spring element or the like.

The hydraulic resistance device having a hydraulic chamber with a piston arranged therein, which divides the hydraulic chamber into an extension chamber and a flexion chamber which are in fluidic communication with one another via the hydraulic line, provides that at least one of the two hydraulic chambers is assigned a nonreturn valve in a throttle valve connected in parallel therewith. The nonreturn valve may always allow the movement in one direction, particularly in the extension movement, while the opposite movement direction of the artificial knee joint is locked when the throttle valve is likewise closed. The switching valve may be connected in parallel with the throttle valve and optionally with the nonreturn valves, so that in a movement direction in which the nonreturn valve is effective hydraulic fluid flows through the throttle valve, optionally in conjunction with the switching valve, and an effective adaptation and increase of the desired resistances may be generated by the throttle valve.

In one embodiment, each hydraulic chamber is assigned a nonreturn valve, the two nonreturn valves being arranged oriented counter to one another and each being connected in parallel with the throttle valve in order to form a bypass in one direction.

One development provides that at least one sensor for recording the flow direction and/or for finding the pressure of the hydraulic fluid is provided. Recording the flow direction can ensure that a swing phase is only initiated by reducing the flexion resistance when the knee joint is loaded in the extension direction and only little in the flexion direction. Recording the flow direction is crucial in order to be able to switch appropriately in the event of loading of the valve body. If an extension is identified by a corresponding flow direction inside the hydraulic line or at a valve, then the switching valve, which may for example be configured as a solenoid valve, is switched as a function of the flow direction. Protection against stumbling may therefore be provided, for example, by keeping the solenoid valve open as soon as it has been enabled so long as the flow direction corresponds to a knee flexion. As soon as the flow changes its direction, the solenoid valve is reclosed. The flow direction may also be recorded by means of status sensors on the nonreturn valves. Providing the protection against stumbling therefore does not require an additional sensor for measuring the knee angle or the knee angular velocity, so that the system complexity is reduced and costs can be saved.

In order to regulate the flow resistance, a throttle valve may be connected fluidically upstream or downstream of the switching valve, the throttle valve or throttle valves being adjustable. The adjustment may take place on the basis of sensor values.

In one embodiment, at least one throttle valve is assigned a second nonreturn valve which is connected in parallel with the first nonreturn valve, is connected upstream or downstream of the throttle valve in the flow direction, and is aligned counter to the first nonreturn valve, the nonreturn valve being assigned a status sensor. The status sensor identifies whether the respective nonreturn valve is opened or closed, so that the flow direction inside the hydraulic line can be detected reliably. Alternatively, the flow direction may also be detected by a flow direction sensor or flow rate sensor provided separately therefor.

At least one relief valve may be arranged in the piston inside the hydraulic chamber, this valve or these valves being arranged respectively in a connecting channel that connects the extension chamber to the flexion chamber. A mechanical overload of the hydraulic resistance device is thereby prevented. Since an overload of the hydraulics is primarily expected in the flexion direction, a relief valve to prevent an excessive pressure in the flexion chamber should be provided in particular. In one embodiment it is provided that two relief valves acting in opposition are arranged respectively in a connecting channel inside the piston in the hydraulic chamber.

The method for controlling an artificial knee joint as described above provides that the resistance is varied as a function of the spatial orientation of the lower part and/or of the upper part, which is determined during use of the artificial knee joint by means of an inertial angle sensor or an IMU, the spatial angle or absolute angle determined being compared with a threshold value and the actuator being activated when the threshold value is reached or exceeded. This makes it possible to achieve reliable control during use of the artificial knee joint on the basis of only one measurement value, namely the spatial angle or absolute angle of a prosthetic component or orthotic component in space, in combination with the flow direction and a loading status, which need not necessarily be detected electronically.

Alternatively or in addition to the spatial orientation, the angular velocity or rate of change of the spatial angle may also be observed, since changes in the movement direction can be detected particularly well by means of these parameters.

Exemplary embodiments of the invention will be explained in more detail below with the aid of the appended figures. Exemplary embodiments with linear hydraulics also apply correspondingly for rotational hydraulics, and vice versa.

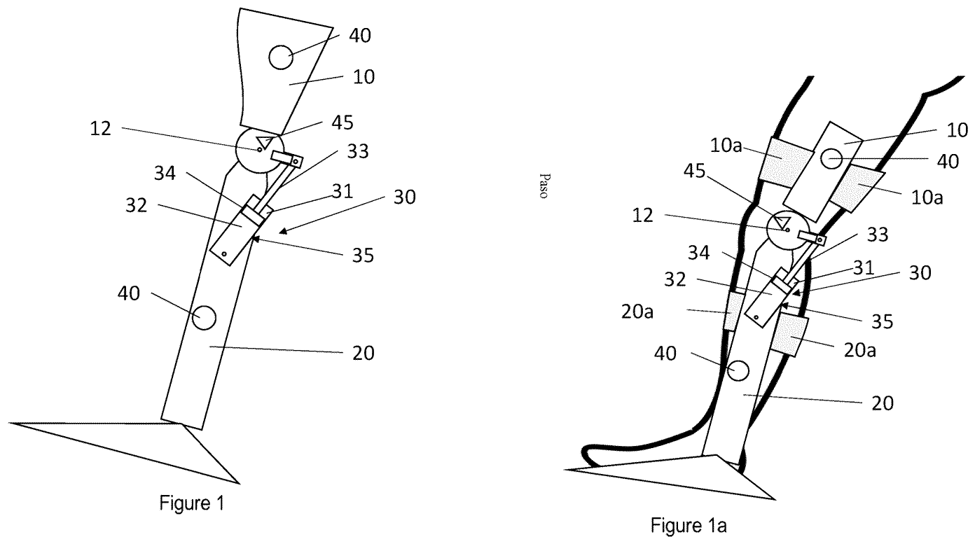

FIG. 1—shows a schematic representation of an artificial knee joint with a resistance device;

FIG. 1a—shows a variant of FIG. 1;

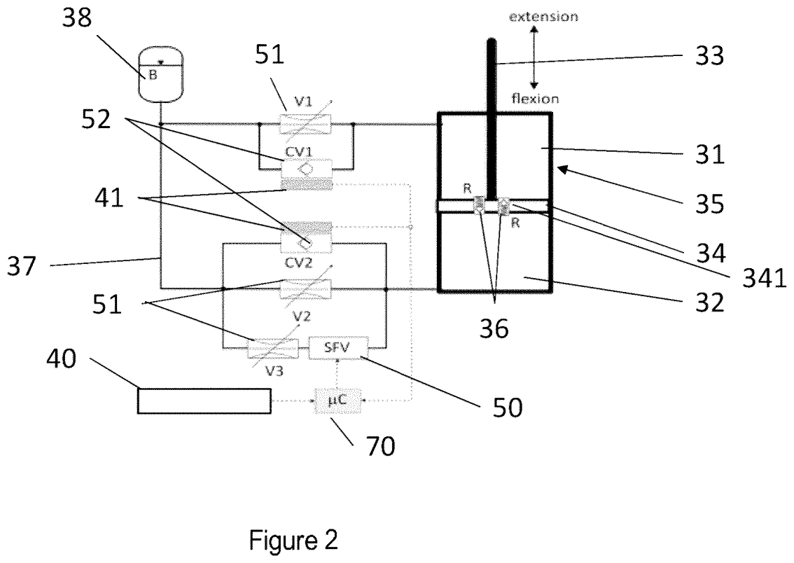

FIG. 2—shows a hydraulic circuit diagram of the resistance device;

FIG. 2a—shows a variant with rotational hydraulics;

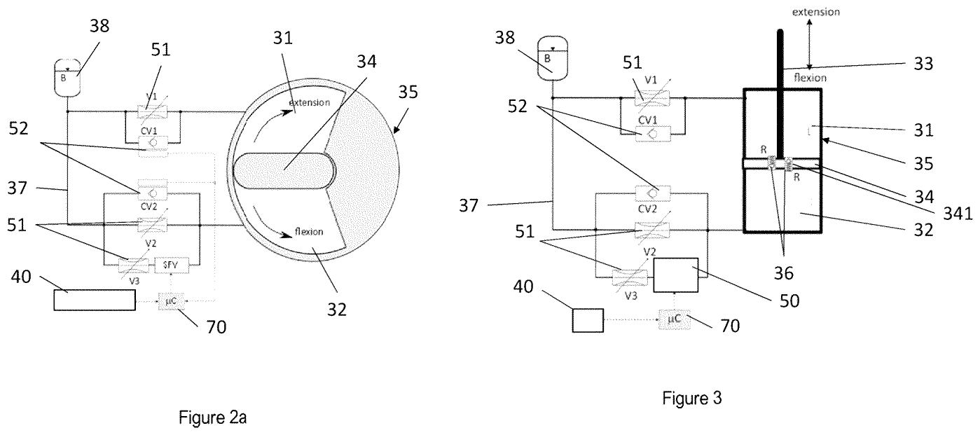

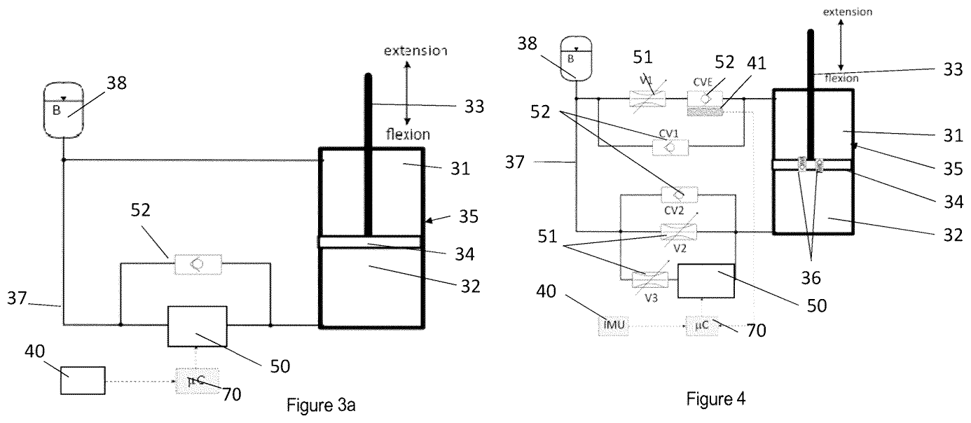

FIG. 3 to FIG. 5—show variants of FIG. 2;

FIG. 6—shows a detail representation of the hydraulic line with a switching valve;

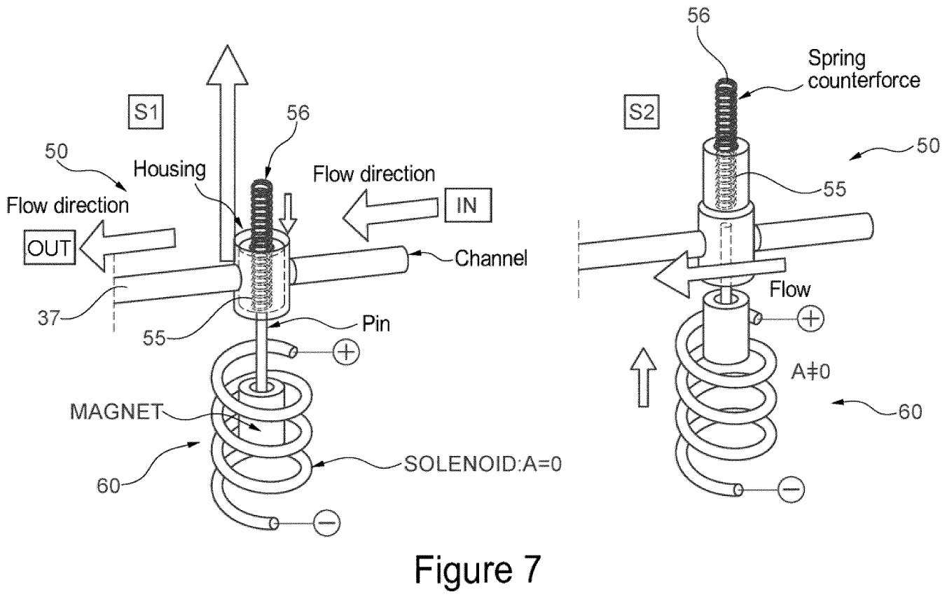

FIG. 7—shows two states of the switching valve and its design structure;

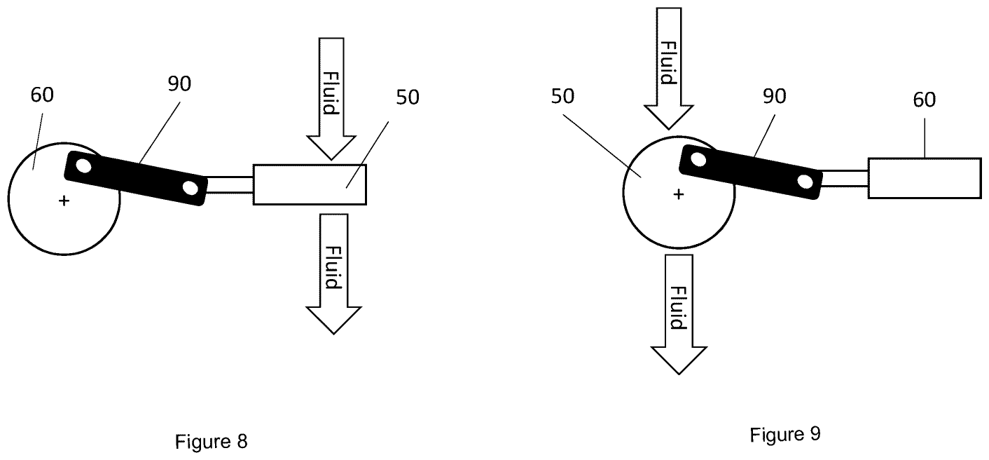

FIG. 8—shows a schematic representation of a linear resistance device with a rotary actuator;

FIG. 9—shows a schematic representation of a rotational resistance device with a linear actuator.

FIGS. 1 and 1a show a schematic representation of an artificial knee joint, as part of a prosthesis in FIG. 1 or an orthosis in FIG. 1a. The artificial knee joint comprises an upper part 10 and a lower part 20, which are mounted on one another so that they can pivot about a pivot axis 12. In an embodiment as the prosthesis according to FIG. 1, a prosthetic foot is arranged on the lower part 20 at the distal end, and in the embodiment of the artificial knee joint as an orthotic knee joint according to FIG. 1a, the lower part 20 may be configured as a lower leg rod, on which a foot part does not need to be arranged. For the case of a KAFO, a foot part on which a foot can be placed is arranged on the lower part 20, as shown. In one embodiment of an orthosis, the upper part 10 is fastened on an upper leg by means of fastening elements 10a and the lower part 20 is fastened on the lower leg by means of fastening elements 20a. The fastening elements 10a, 20a are used for repeated detachable fastening the orthosis on the leg, or the limb. The fastening elements 10a, 20a may be configured as belts, shells, clasps, clips or similar devices in order to fix the orthotic components on the limb in question.

Arranged between the upper part 10 and the lower part 20 there is a resistance device 30 as a linearly acting resistance device 30. In the exemplary embodiment shown, the resistance device 30 is configured as a hydraulic resistance device with a hydraulic chamber 35. The hydraulic chamber 35 is arranged or formed in a housing, and forms a cylinder in which a piston 34 is movably mounted. The piston 34 is movable along the longitudinal extent of the cylinder or of the hydraulic chamber 35, and is fastened on a piston rod 34 which protrudes from the housing or base body with the hydraulic chamber 35 arranged therein. The piston 34 subdivides the hydraulic chambers 35 into an extension chamber 31 and a flexion chamber 32, which are in fluidic communication with one another via the hydraulic line, as will be explained later. The base body or the housing with the hydraulic chamber 35 may be mounted on the lower part 20 so that it can turn, in order to prevent tilting of the piston 34 during a turning movement of the upper part 10 relative to the lower part 20. The end of the piston rod 33 remote from the piston 34 is fastened on the upper part 10, in the exemplary embodiment shown on an bracket in order to increase the distance from the pivot axis 12. During a flexion, the piston 34 is pressed downward so that the flexion chamber 32 is reduced and correspondingly the volume of the extension chamber 31 is increased less the volume of the piston rod 33 being inserted. The differential volume resulting from the piston rod is balanced by a compensating volume (not shown). Because of the flow resistance inside the hydraulic line shown between the extension chamber 31 and the flexion chamber 32, a resistance opposes a flexion movement. The resistance is adjustable. Different volume changes in the extension chamber 31 or flexion chamber 32 are respectively compensated for by means of a compensating volume 38.

In the exemplary embodiment shown, both on the upper part 10 and on the lower part 20 there is arranged a sensor 40 for recording the spatial orientation of the lower part 20 or respectively of the upper part 10. By means of this sensor 40, which may for example be configured as an IMU, the spatial angle or the absolute angle with respect to a fixed spatial orientation, for example the direction of gravity, is determined during use of the artificial knee joint. Instead of an IMU, the sensor 40 may also record other status data, in particular status data that relate to the artificial knee joint. In particular, positions, angular settings, speeds, accelerations, forces and profiles or changes thereof are recorded as status data. The spatial angle determined for the upper part 10 and/or the lower part 20 or another status variable is compared with a threshold angle. When a threshold value that is stored in a control for the respective sensor value or a quantity derived therefrom is reached or exceeded, an actuator is activated or deactivated in order to vary the flow resistance in the resistance device 30.

Alternatively or in addition, a sensor 45 for measuring the knee angle and/or the knee angular velocity may be provided, the information of which may be used in particular to influence the behavior of the resistance device 30 during the swing phase.

The resistance device 30 in an artificial knee joint is used to moderate a flexion movement and an extension movement in order to generate or assist a suitable or desired movement sequence. An extension movement is advantageously braked shortly before reaching a maximum extension, in order to avoid a hard stop. A flexion movement is braked or suppressed in the stance phase and in the swing phase in order to ensure limitation of the bending. Particularly in the stance phase, it is necessary to avoid an erroneous reduction of the flexion resistance after a heel strike in the so-called stance phase flexion. If the artificial knee joint is in a bent setting, for example, and continues to be loaded in the flexion direction, an unintended or undesired reduction of the flexion resistance may lead to unintended bending of the artificial knee joint. In order to achieve reliable use of the artificial knee joint, arranged in the resistance device 30 there is a hydraulic circuit which, by means of mechanical and electronic components, ensures that there is no unintended reduction of a flexion resistance or else of an extension resistance.

FIG. 2 shows the general principle of the hydraulic circuit inside the resistance device 30. The piston rod 33 protrudes from the hydraulic chamber 35 and is directly or indirectly fastened either on the upper part 10 or on the lower part 20 of the artificial knee joint. The hydraulic chamber 35 is then respectively secured on the other part of the artificial knee joint or coupled thereto. It can be seen from FIG. 2 that two relief valves 36 are arranged inside the piston 34 in the connecting channels 341 between the extension chamber 31 and the flexion chamber 32. The two relief valves 36 are arranged acting in opposition in order in the event of an overload not to destroy any mechanical components, but rather to allow yielding against a spring resistance of the relief valves 36. Arranged between the extension chamber 31 and the flexion chamber 32 there is also a hydraulic line 37 through which the hydraulic fluid flows during a movement of the piston 34 when the relief valves are closed. A compensating container 38 is used as a storage container in order to compensate for the volume change due to the insertion or retraction of the piston rod 33. In the exemplary embodiment shown, arranged in the hydraulic line 37 are two nonreturn valves 52, which are oriented acting in opposition. Adjustable throttle valves 51, by means of which the flexion resistance or extension resistance can be adjusted during a flexion movement or extension movement, respectively, are arranged in parallel with the nonreturn valves 52, which may also be spring-loaded. The adjustment may be permanent, or take place once, or it may be varied. In order to achieve a variable throttle effect, the throttle valves 51 are assigned controlling devices which are actuated on the basis of sensor values and/or by means of mechanical force transmission, so that the respective flow resistance in the respective flow direction can be adjusted as a function of sensor values or loads, or settings, in an orthopedic joint device. A switching valve 50 is arranged at the outlet of the flexion chamber 32 in parallel both with a nonreturn valve 52 and with a controlling valve 51. The nonreturn valve 52 at the exit of the flexion chamber 32 locks the fluid flow from the flexion chamber 32 during a flexion movement so that hydraulic fluid from the flexion chamber 32 must flow through the throttle valve 51 and/or the switching valve 50 on the way into the extension chamber 31. In the exemplary embodiment shown, a throttle valve 51 is additionally connected downstream of the switching valve 50 in the flow direction. The switching valve 50 is thus connected to an actuator (not shown), which is in turn activated or deactivated by means of a control device 70. The control device 70 is coupled to one or more outer sensors 40 for recording status data, and activates or deactivate the actuator in order to open or close the switching valve 50. Besides the external sensors 40, status sensors 41 of the nonreturn valves 52 are optionally coupled to the control device 70. The status sensors 41 in their simplest embodiment are configured as switches and detect whether the respective nonreturn valve 52 is opened or closed. If the nonreturn valve 52 assigned to the switching valve 50 is in a closed state in order to lock the fluid flow during a flexion movement, it can be detected thereby which movement or which load is applied to the artificial knee joint. If this nonreturn valve 52 is in an opened setting, it can be seen thereby that no moment that would cause a flexion movement is acting about the pivot axis 12. This is essential input information for the control device 70.

FIG. 2a shows a variant of FIG. 2 with rotational hydraulics. The hydraulic circuit connecting the extension chamber 31 to the flexion chamber 32 corresponds to the circuit according to FIG. 2. Instead of a linearly acting piston, a rotary piston 34 that separates the extension chamber 31 from the flexion chamber 32 is arranged inside the hydraulic chamber 35. The compensating container 38 is used to compensate for fluid losses and thermally induced changes of the oil volume. The compensating container 38 is not necessary in order to compensate for different volumes due to a piston rod being inserted or retracted since the latter is absent in rotational hydraulics. The compensation volume therefore becomes comparatively small in the case of rotational hydraulics compared with linear hydraulics.

FIG. 3 shows a variant of the embodiment according to FIG. 2 in which there is essentially the same structure but the control device 70 is not equipped with sensors 41 for recording the flow direction, or for finding the status of the nonreturn valves 52. Rather, with a substantially unmodified mechanical structure, simplified control is provided merely on the basis of status data by means of the sensors 40, for example by means of an IMU and exclusively only an IMU. The switching valve 50 is configured in such a way that a release force exerted by the actuator in order to enable the flow channel does not exceed an adjusted setpoint value. The setpoint value is ascertained by the fact that, with a predefined pressure force, a valve body inside the switching valve 50 requires a predefined release force, that is to say a resistance opposes an adaptation from a closed state into an opened state. If a knee joint is loaded with a force or a moment that acts in the flexion direction, a pressure force due to the hydraulic pressure from the flexion chamber is applied both to the throttle valve 51 connected in parallel and to the switching valve 50. The nonreturn valve 52 likewise connected in parallel at the flexion chamber outlet is closed. If in such a state the control device 70 receives from a sensor device 40 a sensor value which, after evaluation, leads to a control instruction that brings about activation of the actuator, a corresponding signal is output to the actuator and a release force is exerted on the valve body. If the holding force opposing the release force due to the applied pressure is too great, the switching valve 50 cannot open so that erroneous initiation in the event of a knee joint loaded in the flexion direction is prevented.

The switching valve 50 may be designed so that, after activation once by the control device 70, it remains open until the flow of fluid through the valve falls below a threshold value. Unimpeded flexion of the knee joint may therefore be achieved, but the knee joint device automatically returns into the highly damped, safe state as soon as the movement is ended or the movement direction is reversed.

FIG. 3a shows the minimum equipment of a resistance device with the hydraulic chamber 35 and the piston 34 arranged therein in order to form an extension chamber 31 and a flexion chamber 32. Alternatively to a configuration as linear hydraulics, it may also be configured as rotational hydraulics according to FIG. 2a. The hydraulic line 37 connects the extension chamber 31 to the flexion chamber 32 and the compensating volume 38. Before a connection of the hydraulic line 37 to the flexion chamber 32 there are a nonreturn valve 52 and a switching valve connected in parallel therewith. The switching valve 50 is adapted by means of the control device 70, which is in turn coupled to the sensor device 40. The nonreturn valve locks the outflow from the flexion chamber 32 in the direction of the extension chamber 31 and still enables the flow from the extension chamber 31 into the flexion chamber 32. It is therefore possible that extension can always take place, but flexion can only take place if the switching valve 50 is correspondingly opened.

In FIG. 4 there is a similar hydraulic structure as in FIGS. 2 and 3, but the extension chamber 31 is assigned at the exit a further nonreturn valve 52, which is in turn assigned a status sensor 41 for recording the flow direction. Two nonreturn valves 52 connected in opposition are therefore arranged at the exit of the extension chamber. If an extension movement takes place, the status sensor 41 detects an opened nonreturn valve and can thereby determine the movement direction and the loading direction in the artificial knee joint. In the case of a flexion movement, the closed state of the nonreturn valve 52 provided with the status sensor 41 can be detected. If in this structure the switching valve 50 is opened and furthermore a hydraulic flow is possible through the throttle valve or throttle valves 51, then a return flow into the extension chamber 31 is also possible.

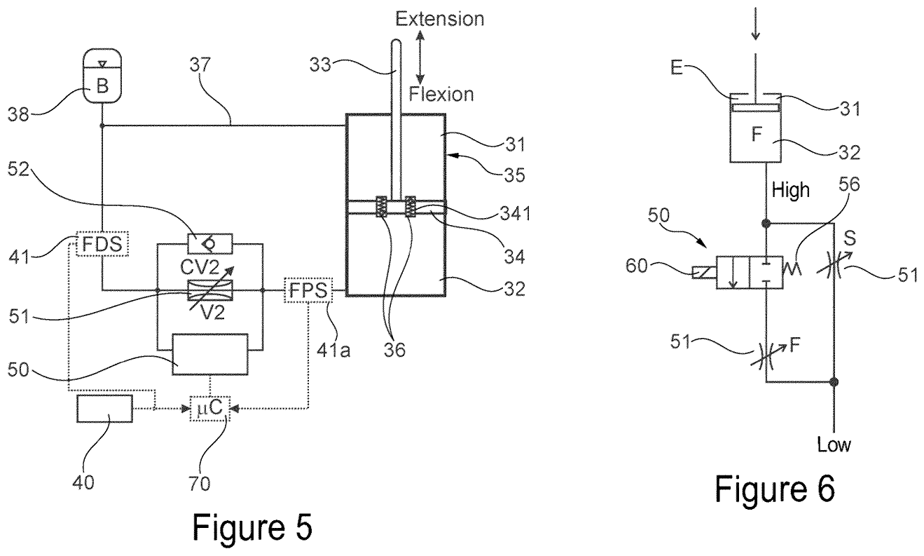

Shown in FIG. 5 is a variant of the hydraulic structure of the resistance device in which a flow-direction, flow-rate or flow sensor 41, which is coupled to the control device 70, is arranged in the hydraulic line 70 from the extension chamber 31 to the flexion chamber 32. A pressure sensor 41a, which is optional, is arranged between the switching valve 50, the throttle valve 51 and the single nonreturn valve 52 in parallel with the two aforementioned valves 50, 51, in order to ensure that no bending load, force or moment is applied to the knee joint in the flexion direction. The pressure sensor 41a may be constructed simply, since only a binary or digital status signal is necessary, so that the pressure sensor 41a may also be configured as a pressure switch that is activated by a piston or a membrane when a sufficiently high hydraulic pressure from the flexion chamber 52 is applied in the hydraulic line 37. The direction sensor 41 for identifying the flow direction or pressure direction of the hydraulic fluid may have a different design, for example as a nonreturn valve with status recording, as a turbine wheel with rotation direction recording, as a gauge sensor, as a light beam sensor, by means of magnets, or the like. The throttle valve 51 in parallel with the switching valve 50 is generally a valve with a high throttle effect, which is used for example for stance phase flexion damping. The throttle valve 51 shown in FIGS. 2 to 4, which is assigned to the flexion chamber 32, is used for extension damping in the case of a flow of the hydraulic fluid from the flexion chamber 32 back into the extension chamber 31, and has a comparatively low throttle effect which may also be adapted as a function of the position of the piston 34. The throttle valve arranged in the flow direction from the flexion chamber 32 into the extension chamber 31, behind the switching valve 50 in the flow direction, is used for flexion damping in the swing phase and is an optional valve, which has a comparatively low throttle effect that may likewise be adapted as a function of the position of the piston 34.

FIG. 6 schematically shows a more detailed representation of the switching valve 50 and an actuator 60. The hydraulic structure is indicated only incompletely, a return line from the flexion chamber 32 to the extension chamber 31, as well as a compensating container, being omitted. In the state shown, the switching valve 50 is closed, i.e. fluid from the flexion chamber 32 cannot flow through the switching valve 50. It has to pass through the throttle valve 51 connected in parallel with a very high throttle effect in order to reach the extension chamber 31. The switching valve 50 is assigned an actuator 60, which is configured as a motor, electromagnet, solenoid or other switching device or actuation device, in order to be activated on the basis of sensor values by means of the control device 70 (not shown). In the exemplary embodiment shown, the actuator 60 acts against an energy storage mechanism 56, for example a spring, an elastomer element or a magnet. If the actuator 60 is not activated, the switching valve 50 is closed. In order to open the switching valve 50 and reduce the flexion resistance, the actuator 60 is activated and a force is applied sufficiently to move an unloaded switching valve 50 from the closed setting shown into an opened setting, or release setting, when there is no pressure force component acting on a valve body due to an applied hydraulic pressure. If however a pressure is applied to the valve body of the switching valve 50 because of a force (which is indicated by an arrow) acting on the piston rod, a holding force is thereby generated, for example by the pressing of the valve body into its guide, so that the release force applied by the actuator 60 is not sufficient to move the valve body from the closed setting into the release setting. The throttle valve 51 optionally connected in series with the switching valve has a rather low throttle effect and is used to adjust the swing phase damping.

FIG. 7 shows the schematic structure of the switching valve with the valve body 55 inside a valve housing with the drive 60 and the spring element 56. In the representation on the left, the hydraulic flow inside a hydraulic line is blocked. The valve body 55 is located in a housing in which the hydraulic line 37 is arranged or formed. The valve body 55 is assigned via the actuator 60 in the form of a solenoid or a magnet coil, inside the coil there is a magnet that is coupled to a pin which in turn acts on the valve body 55. If a current flows through the coil, the magnet moves upward inside the coil and the pin presses on the valve body 55 against the spring force that is applied by the energy storage mechanism 56 and holds the valve body 55 in the first, closed setting. This state is shown in the representation on the right. The hydraulic line 37 is fully opened and allows the hydraulic fluid to flow through. The mechanical interaction between the valve body and the housing is configured so that, when a hydraulic pressure is applied from the flexion chamber in the direction of the extension chamber, friction or engagement of projections and recesses takes place so that an increased displacement force is necessary in order to move the valve body 55 into the opened setting. This pressure force is sufficient to increase the required release force beyond an amount such that the necessary force cannot be applied by the actuator 60. Thus, if a hydraulic pressure is applied to the valve body 55, the actuator 60 cannot move the valve body 55 into the second, opened setting against the spring force of the energy storage mechanism 56 in combination with a holding force by the mechanical mounting or a corresponding configuration of the incident flow surface of the valve body 55. The housing, or the housing and the valve body 55, is/are advantageously configured so that a high release force, which has to be applied by the actuator 60, is necessary in the event of a high pressure.

With the mechanical design of the resistance device 30 in combination with the control concept of the control device 50, it is possible to make do with a minimum of sensor signals for controlling a flexion release and/or an extension release. This may for example via a single spatial attitude sensor 40 or an IMU information relating to a swing phase release be sent to the control device 70, by means of which an actuation of the actuator 60 and a subsequent release of the switching valve 50 would normally take place. If however a sufficiently large pressure force component is applied perpendicularly to the displacement direction of the valve body 55, for example because of an unforeseen flexion load, the necessary release force is increased due to the mechanical design so that the release cannot take place. Only when the pressure force from the flexion chamber 32 abates can the switching valve 50 be opened, so that the bypass for the conventional throttle valve 51 is opened and the hydraulic resistance is reduced. In the return circuits mentioned above, an extension movement is always possibly because of the nonreturn valves. In principle, it is therefore possible to enable reliable and in particular safe control of an artificial knee joint exclusively with an IMU. In particular, unintended initiation of a flexion movement is avoided.

FIG. 8 shows the linking of a switching valve to a lever gearing via a rotational drive 60. The rotary drive 60, or rotational drive, may be configured as a motor or as a rotary component of a gearing device connected downstream of a motor. By means of a lever 90, which is mounted or fastened so that it can pivot both on the rotary drive 60 and on the linear switching valve 50, the adaptation of the valve 50 can be brought about in the manner of a connecting rod.

Alternatively, a linearly actuated switching valve may also be actuated directly by means of a linear drive. Transformation may in this case be produced by means of an optional lever mechanism.

FIG. 9 shows the kinematic reverse of FIG. 8, in which there is a linear drive 60 that is coupled by means of a lever 90 corresponding to the arrangement of FIG. 8 to a switching valve 50 configured as a rotary valve. The respective flow direction and incident flow of the hydraulic fluid are represented by the arrows.

Alternatively, a switching valve configured as a rotary valve may also be actuated by means of a rotational actuator. Besides a direct drive, the actuation may also take place via gearing with suitable transformation, a link rod mechanism or a combination of these components.

1. An artificial knee joint, comprising:
an upper part; and
a lower part, wherein the upper part and the lower part are mounted on one another so that they are pivotable about a pivot axis;
a hydraulic resistance device between the upper part and the lower part configured to provide a resistance to a pivoting movement;
a switching valve in a hydraulic line of the hydraulic resistance device, wherein, the switching device comprises a valve body which is displaceable in a displacement direction, wherein the switching valve blocks or partially closes the hydraulic line in a first setting and releases the hydraulic line in a second setting, and wherein the switching valve is configured or arranged such that a pressure force component acting on a valve body of the switching valve perpendicularly to the displacement direction as a result of hydraulic fluid generates a holding force which opposes a displacement of the valve body;
an actuator configured for exerting a release force which moves the valve body out of the first setting and into the second setting is assigned to the valve body, wherein the actuator is coupled or coupleable to a control device which is connected to a sensor for recording status data, wherein the control device activates the actuator based on the status data, and wherein the release force is adjusted to be less than the holding force for a predefined pressure force.

2. The artificial knee joint as claimed in claim 1, wherein the actuator is configured as a motor or electromagnet.

3. The artificial knee joint as claimed in claim 1 wherein the valve body is assigned an energy storage mechanism and/or a magnet which opposes the release force.

4. The artificial knee joint as claimed in claim 3, wherein the energy storage mechanism is a spring element/energy storage mechanism that is configured to be adaptable/adjustable.

5. The artificial knee joint as claimed in claim 1 wherein the release force that can be applied by the actuator is adjustable.

6. The artificial knee joint as claimed in claim 1 further comprising a gearing, a lever mechanism, or a link rod combination, is arranged between the actuator and the valve body.

7. The artificial knee joint as claimed in claim 1 wherein the hydraulic resistance device comprises a hydraulic chamber with a piston arranged therein, wherein the piston subdivides the hydraulic chamber into an extension chamber and a flexion chamber which are in fluidic communication with one another via the hydraulic line, wherein at least one of the extension chamber and the flexion chamber is or are assigned at least one nonreturn valve with at least one throttle valve connected in parallel with the at least one nonreturn valve.

8. The artificial knee joint as claimed in claim 7, wherein the switching valve is connected in parallel with the at least one throttle valve.

9. The artificial knee joint as claimed in claim 7 wherein the at least one nonreturn valve includes at least two nonreturn valves, and wherein each of the extension chamber and the flexion chamber is assigned a nonreturn valve of the at least two nonreturn valves, and wherein and the at least two nonreturn valves are arranged acting in opposition.

10. The artificial knee joint as claimed in claim 7 further comprising at least one for recording a flow direction and/or for recording pressure.

11. The artificial knee joint as claimed in claim 7 further comprising a further throttle valve is connected fluidically upstream or downstream of the switching valve.

12. The artificial knee joint as claimed in claim 7 wherein the at least one throttle valve is adjustable.

13. The artificial knee joint as claimed in claim 7 wherein the at least one nonreturn valve comprises a first nonreturn valve and a second nonreturn valve, and wherein the at least one throttle valve is assigned the a second nonreturn valve connected in parallel with the first nonreturn valve, wherein the second nonreturn valve is connected upstream or downstream of the at least one throttle valve in the flow direction, and wherein the second nonreturn valve acts in opposition to the first nonreturn valve, and is assigned a status sensor.

14. The artificial knee joint as claimed in claim 7 further comprising at least one relief valve is arranged in the piston, wherein the at least on relief valve is arranged in a connecting channel that connects the flexion chamber to the extension chamber.

15. The artificial knee joint as claimed in claim 7 wherein the at least one nonreturn valve is assigned a sensor for status recording of the at least one nonreturn valve.

16. A method for controlling an artificial knee joint as claimed in claim 1, comprising:
varying resistance as a function of a spatial orientation of the lower part and/or of the upper part, wherein the spatial orientation includes a spatial angle and is determined during use of the artificial knee joint by an inertial angle sensor, wherein the spatial angle determined is compared with at least one threshold value; and
activating or deactivating the actuator when the threshold value is reached or exceeded.

17. A method for controlling an artificial knee joint as claimed in claim 1, comprising:
varying resistance a function of changes in a spatial orientation of the lower part and/or of the upper part, wherein the spatial orientation is determined during use of the artificial knee joint by an inertial angle sensor, wherein a rate of change of the spatial orientation determined is compared with at least one threshold value; and
activation or deactivating the actuator when the threshold value is reached or exceeded.

18. The method as claimed in claim 17, wherein, because of the fluid properties of the switching valve, a flexion of an artificial knee joint initiated after activation of the actuator can be performed unimpeded until the natural movement reversal of the knee joint.

19. The method as claimed in claim 17, wherein, because of the properties of the switching valve, after activation of the actuator, the flexion of an artificial knee joint can only be initiated if the bending moment at the time of activation of the actuator does not exceed a defined threshold value.

20. The method as claimed in claim 19, wherein the threshold value is negative, which corresponds to an extension moment at the artificial knee joint.
